(19) United States
(12) Patent Application Publication
(10) Pub. No.: US 2008/0202531 A1
(43) Pub. Date: Aug. 28, 2008

(54) Protective Aid

(76) Inventor: Robyn Maria Fletcher, Hamilton (NZ)

Correspondence Address: YOUNG & THOMPSON, 209 Madison Street, Suite 500, ALEXANDRIA, VA, 22314, US

(21) Appl. No.: 11/916,316

(22) PCT Filed: Jun. 2, 2006
(86) PCT No.: PCT/NZ06/00139

§ 371 Date: Dec. 3, 2007

(30) Foreign Application Priority Data
Jun. 2, 2005 (NZ) 536934
Jun. 2, 2006 (NZ) 547746

(51) Int. Cl. A61F 13/00

(52) U.S. Cl. 128/888; 128/846

(57) ABSTRACT

This invention relates to a protective aid (1), which is applicable generally in the field of sanitary items. The aid has application in conjunction with a range of covering articles, such as nappies, breast pads, wound dressings and the like. The protective aid includes a body having an internal (3) and external surface (2). The body is adapted to include a defined cavity (5) such that a substantial portion of the internal surface of the aid is distanced from an area being protected by the aid. The body, also includes an external rim (4) contactable with a surface adjacent the area being protected. The protective aid is also adapted to receive securing means (9) on the body (at 8) for securing the body relative to either or both the area being protected and a covering article.

### TECHNICAL FIELD

The present invention relates to a protective aid, previously entitled “Umbilical Aid”.

In particular, the present invention relates to an aid which is applicable generally in the field of sanitary items.

It is envisaged the aid will have application in conjunction with other articles, such as nappies, breast pads, wound dressings and the like. However, the present invention may have applications outside this field.

### BACKGROUND ART

During the development of an improvement in and relating to a nappy for use with newborn babies (NZ Patent No. 513119), the applicant identified problems associated with the use of nappies on the healing, comfort and accommodation of the umbilicus. The applicant noted that the newly severed residue umbilical cord provides a potential infection and/or inflammation site and that use of inappropriately designed or sized nappies can lead to numerous difficulties such as:

frictional irritation of the umbilical cord by direct contact with the nappy liner; or

if the nappy does not cover the umbilical cord, frictional irritation of the umbilical cord by contact with the garments clothing the infant; and/or

transmission of faeces and urine to the umbilical region via a body movement of the infant in the absence of correctly sized/designed physical barriers within the nappy lining.

The current thinking relating to the treatment of the umbilical cord site with respect to sanitary nappies was also identified. The discussion identified two main schools of thought. On the one hand, the umbilical cord is left uncovered by the nappy. Alternatively, the umbilical cord and surrounding portion of the infant's torso is covered by a relatively large nappy. The discussion relating to these two schools of thought is reiterated here.

It was noted that leaving the umbilical cord uncovered accelerates the time required for the cord to dry out and detach from the umbilicus. Various prior art solutions following this philosophy were introduced, such as U.S. Pat. No. 4,230,113, (Mehta) where v-shaped, or u-shaped notches were included in the upper front portion of the nappy to leave the umbilical cord exposed. U.S. Pat. No. 4,675,015 (Brown) incorporated a separable opening through which the umbilical cord could project.

However, such nappy configurations were viewed as not obviating problems of the lack of protection of the clamped umbilical cord from being snagged on the infant's clothing, or from unintentional impact by the infant or caregiver during handling of the child.

The alternative approach of covering the umbilicus with a conventionally designed nappy was identified as also being fraught with undesirable drawbacks. In order to adequately cover the entire umbilical area (including the un-detached umbilical cord and clamp) and provide a sufficient margin with the nappy perimeter to prevent any protrusion of the cord, it was identified that a nappy dimensioned with a disproportional high-front section was required. In practice, nappies sold as suitable for newborn infants are simply scaled-down versions of larger conventional proportioned nappies and so nappies with a high-front section are not available.

Consequently, the un-detached umbilical cord is either enclosed by the nappy beneath the nappy liner surface, or the upper front section of the nappy is folded (inwards or outwards) to expose the umbilical cord as per the former approach.

Neither approach was deemed to adequately protect the umbilicus from infection, irritation, abrasion and trauma. It was considered that, any discomfort experienced by the infant as a consequence of these defects will cause unwanted anxiety in both the infant and caregivers.

Having regards to the problems of protecting the umbilicus from infection, irritation, abrasion and trauma, it became apparent that protection of the umbilicus is not the only area where an invention directed to addressing problems in the prior art could have potentially realisable advantages.

For example, sensitive nipples of breast feeding women also present comparable issues due to frictional irritation by direct contact with breast pads, bra liners, or bra material; and comparable issues relating to hygiene exist where there is risk of infection of nipples and breast tissue.

Further, similar problems are encountered when treating wounds, where frictional irritation and issues of hygiene are equally relevant to the healing process and where treatments echo those previously discussed in relation to the umbilical cord. Accordingly, a wound may be left uncovered or covered by an appropriate wound dressing. Of relevance to newborn babies is the issue of circumcision, where typical dressings or nappies are not adapted to minimise infection, irritation, abrasion and trauma. Other instances generally would include bums, areas which have been newly operated on and the like.

The applicant also appreciates there are likely to be other situations where similar factors impact on protection of a site from infection, irritation, abrasion and trauma, which may not adequately be catered for by prior art systems.

While the present invention has a number of potentially realisable applications, it is in relation to problems associated with the protection of sites on the body of a human or other animal from infection, irritation, abrasion and trauma while healing/recover can take place, that the present invention was developed. More specifically, it was with safety and health issues associated with the protection of such sites, that the present invention was developed.

It would be useful therefore, to have an aid for use in the protection of sites on the body of a human or other animal that:

a) Was able to be used in a manner which had the potential to minimise irritation from surrounding clothing/covers; and/or

b) Was able to be used in a manner which had the potential to minimise abrasion from surrounding clothing/covers; and/or

c) Was able to be used in a manner which had the potential to minimise

d) Was able to be used in a manner which had the potential to minimise infection or assist recovery; and/or

e) Would be able to be used in confined spaces; and/or

f) Would be easy to use; yet

g) Could be used with the benefits of safety and health issues in mind; and

h) Could offer an alternative to existing systems and/or

i) Could be disposable or reusable, depending on its application.

It would therefore be advantageous to have an invention that offered at least some, if not all, of the advantages of the above proposed aid.

It is therefore an object of the present invention to consider the abovementioned problems and provide at least one solution which addresses one or more of these problems.

It is a specific objective of the invention to provide comfort and safety in the form of an aid that can be positioned and formed in such a way as to protect the umbilical cord/clamp said navel area from irritation.

It is a specific objective of the invention to provide comfort and safety in the form of an aid that can be positioned and formed in such a way as to protect the nipple area, of breast feeding women, from irritation.

It is a specific objective of the invention to provide comfort and safety in the form of an aid that can be positioned and formed in such a way as to protect a wound area from irritation.

It is also an object of the present invention to at least provide the public with a useful choice or alternative system.

A further object of the present invention is to offer an aid which may also be cost effective to produce and therefore be an economical alternative to existing apparatus or methods.

Further aspects and advantages of the present invention will become apparent from the ensuing description which is given by way of example only. It should be appreciated that variations to the described embodiments are possible and would fall within the scope of the present invention.

This specification is also based on the understanding of the inventor regarding the prior art. The prior art description should not be regarded as being an authoritative disclosure of the true state of the prior art but rather as referring to considerations in and brought to the mind and attention of the inventor when developing this invention. Discussions relating to any references merely reiterate what their authors assert, and the relevance or accuracy of the cited documents is not absolute, but simply raised within a general discussion of potentially useful information.

### DISCLOSURE OF INVENTION

The umbilical cord is the primary support vehicle for transmission of blood and nutrients from the mother to the foetus. At birth the umbilical cord is severed leaving the newborn baby to sustain itself. Remnants of the umbilical cord gradually involutes and regress forming the navel, commonly knows as the “belly button”.

This process occurs in days or sometimes weeks.

The proximity of the navel falls directly around the front lower stomach area that is adjacent to the region around which a nappy is wrapped and firmly held. There is typically a risk of irritation from disposable nappies to this sensitive region.

Similarly, the nipple is the primary vehicle for breast feeding a baby. There is typically a risk of irritation through cracked nipples and possible infection to this sensitive region.

A wound is defined as any break in the skin or an organ or part as the result of an injury or from surgical intervention. Again such areas are sensitive and prone to infection.

The aid, whether as an umbilical aid for babies wearing nappies, as a wound aid, as a breast nipple aid, or any other appropriate aid, is a protective cover.

According to one aspect of the present invention, there is provided a protective aid, said protective aid protective aid including a body, said body having an internal and an external surface, said body adapted to include a defined cavity such that a substantial portion of the internal surface of the aid is distanced from an area being protected by the aid to accommodate said area, said body also including an external perimeter/rim contactable with a surface adjacent the area being protected, said protective aid also adapted to receive securing means on the body for securing the body relative to either or both the area being protected and a covering article.

In preferred embodiments of the present invention the aid is positioned over or relative to an area requiring protection. Accordingly, depending on its application, the protective aid will be differently configured. The shape and size for use in protecting an umbilical cord, may be different from the shape protecting a nipple or breast; and different from the range of shapes which may be relevant to wounds. Of course, the configuration of the area adjacent the protected area is also relevant as it may impact on the configuration of the aid in order that the aid fit more comfortably.

Typically the body of the aid may be held in position directly by a covering article. For example, where the protective aid is used to protect the umbilical cord, the article may be the baby's nappy, or an article of clothing, and so forth; where the protective aid is used to protect a wound, the article may be a bandage or section of an adhesive strip; where the protective aid is used to protect the nipple, the article may be a bra, or other article of clothing.

However, in other embodiments, the aid may be adapted to receive securing means which are independent from the covering article, but attachable both to the aid and an article. The securing means may limit movement of the protective aid that may occur when the person or animal moves. The securing means may include any means available in the prior art or adapted for use with the protective aid. Examples include, hook and pile systems, material ties, press studs, buttons, hooks and so forth. The choice of securing means is dictated by the application and the requirements of each case—having consideration for minimising discomfort, retaining the aid effectively in place, the area being covered, being maintained free of or minimising ingress by fluids; and so forth.

In embodiments which may be applied for use to protect an umbilicus, the securing means may be a hook that is layered between layers of material from which the aid is made. For example, a hooking means may be layered between the top (outer) and a second (inner) layer of fabric. The hook is preferably adapted to grab or hook on to the inside of the nappy.

Where the aid is to be used in conjunction with a nappy a preferred fabric may be used to keep the nappy aid positioned. The nappy aid is positioned directly over the umbilical cord enclosing the umbilical cord and cord clamp. Conveniently to prevent movement the preferred fabric on the outer nappy aid connects to the inside of the disposable nappy fabric enabling it to stay secure and positioned that is securing the umbilical cord and clamp navel region. Conveniently the nappy aid is disposed of but fabric/materials may enable it to be other wise.

Alternatively, in some embodiments where firmer retention is required and/or a fluid tight seal is required to prevent ingress into the protected area of fluids or contaminating solids, the aid may include around its edges/rim an adhesive layer to stick the protective aid to the surface of the skin in a temporary manner. Preferably, the adhesive layer with be applied using anti-allergenic adhesive materials. Preferably the adhesive used will enable easy removal of the aid when required, without causing discomfort or pain.

The aid is preferably formed from breathable materials with smooth, non-stick, non-irritating surfaces, particularly the internal surface. Accordingly, the aid may be made from thermoplastics materials, or fabrics which meet the required criteria, or from a combination of materials. It may be configured to be made from obviously porous materials, or from materials which are less porous, but are adapted to maintain conditions internally of the aid to an optimum level.

Preferably, the aid is formed from fabric using the art of layering to achieve a degree of strengthening to maintain a preferred shape and rise to the aid to help protect the umbilical cord/clamp, a wound, a nipple and so forth; and to prevent external pressure on the outer interferences and surrounds. The layering effect may also be achieved by having one material as an external surface with different materials layered therebeneath. For example, the aid may comprise an outer layer of thermoplastic material, with subsequent internal layers such as a soft layer, a non-stick layer, or a therapeutic layer—any one of which may be adjacent the area being protected.

The aid may be pre-pressed into, pre-moulded to, or be mouldable to the desired shape for use. The materials from which the aid is made will influence the way the configuration of the aid is achieved. For example, some mouldable materials, such as certain thermoplastics, may be treated with hot water and moulded to a desired shape before use.

An inner layer of the aid or the entire aid may be impregnated with, or capable of receiving, an application directed to facilitating healing, minimising infection, or providing a cooling/anti-inflammatory effect and so forth. Accordingly, the application may include a sanitising, antiseptic, antimicrobial, anti-inflammatory preparation and so forth, applied to or within the layer closest to the area being protected. The application may or may not contact the area being protected.

Preferably the aid is lightweight to minimise discomfort and weight.

It will be appreciated that the invention broadly consists in the parts, elements and features described in this specification, and is deemed to include any equivalents known in the art which, if substituted for the prescribed integers, would not materially alter the substance of the invention.

Variations to the dimensions of the protective aid may also be desirable depending on the purpose for which it is to be used.

The present invention is differentiated from many existing products by virtue of at least its configuration which distances the protective aid from the area being protected; as a result of the aid being integral with a covering article, or attachable to a covering article, or merely being held in place by a covering article.

Whilst some varying embodiments of the present invention have been described, it should further be appreciated different embodiments, uses, and applications of the present invention also exist.

An embodiment of the present invention will now be given by way of example only, to help better describe and define the present invention.

However, describing the specified embodiment should not be seen as limiting the scope of this invention.

### BRIEF DESCRIPTION OF DRAWINGS

Further aspects of the present invention will become apparent from the following description, given by way of example only and with reference to the accompanying drawings in which:

FIG. 1a is a side view of a protective aid in accordance with one preferred embodiment of the present invention; and

FIG. 1b is a top view of a protective aid of FIG. 1a in accordance with that preferred embodiment of the present invention; and

FIG. 1c is a top perspective view of the outer surface a protective aid of FIG. 1a in accordance with that preferred embodiment of the present invention; and

FIG. 1d is a perspective view showing the inner surface a protective aid of FIG. 1a in accordance with that preferred embodiment of the present invention.

### BEST MODES FOR CARRYING OUT THE INVENTION

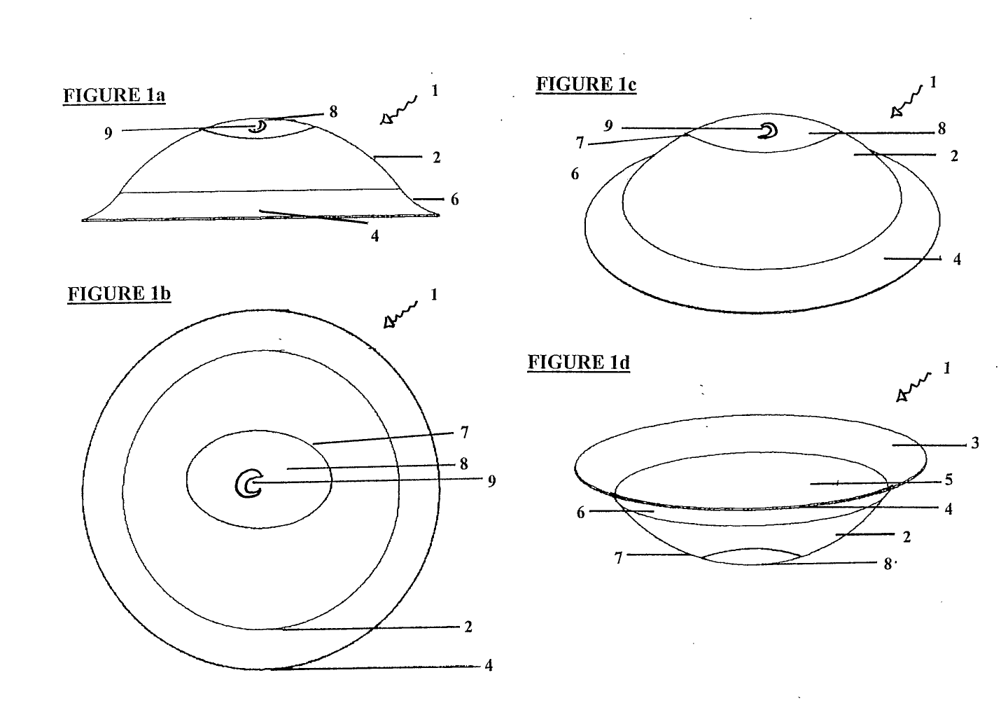

With reference to the diagrams (FIGS. 1a to 1e), by way of example only, there is provided a protective aid (generally indicated by arrow (1)). Whilst in this description the protective aid is described herein with reference to an umbilical protective aid, it should be appreciated this invention has applications outside this field.

The protective aid in FIGS. 1a to 1c, is substantially circular and hemi-spherical. The protective aid includes an external surface (2), internal surface (3) and a rim (4). The aid is also substantially concave to form an internal cavity (5) as seen in FIG. 1d, to create an area capable of accommodating an area to be protected thereby. It should be appreciated that different aids for different uses will be differently configured.

In this embodiment, the aid is used to provide a moderately sturdy cover over the remnants of an umbilical cord, and by doing so assists in preventing direct contact of the umbilicus with the baby's clothing or nappy. The aid is configured to adequately cover the entire umbilical area (including the un-detached umbilical cord and clamp) and provide a sufficient margin with the nappy perimeter to prevent any protrusion of the cord.

The rim (4) of the aid is substantially tapered (at (6) as shown in FIG. 1d, which may assist in providing a more stable positioning of the aid over the area, without introducing a significant contact region between it and the baby's skin where moisture may collect and cause chaffing and so forth.

The size, shape and general configuration of the protective umbilical aid is important for minimising potential infection and/or inflammation of the site, as well as minimising frictional irritation of the umbilical cord by direct contact with the nappy liner; or frictional irritation of the umbilical cord by contact with the garments clothing the infant; and/or transmission of faeces and urine to the umbilical region via a body movement of the infant.

The upper region (7), of the external surface of the aid includes an area (8) where securing means may be accessible from or attachable to the aid, to secure the aid in position relative to an article of clothing or an internal surface of a nappy.

The securing means in this embodiment is a hooking means (9) for hooking the aid in position relative to an article of clothing or an internal surface of a nappy.

It should be appreciated that the present invention lends itself to more than one application. Hence, the invention may be adapted through a change to one or more of any structural feature, dimensions, and so forth. As can be appreciated a variety of different embodiments, uses, and applications of the present invention therefore exist, even within the ambit of the above described system.

As previously described, various embodiments may vary in the manner of construction and operation, but yet may achieve the same outcome.

Therefore, the protective aid of the present invention has potentially realisable benefits over prior art systems in that it provides as listed below, an aid with the following features which may be applied singularly or a combination as required—an aid for use in the protection of sites on the body of a human or other animal that:

a) May be used in a manner which had the potential to minimise irritation from surrounding clothing/covers; and/or

b) May be used in a manner which had the potential to minimise abrasion from surrounding clothing/covers; and/or

c) May be used in a manner which had the potential to minimise

d) May be used in a manner which had the potential to minimise infection or assist recovery; and/or

e) May be able to be used in confined spaces; and/or

f) May be easy to use; yet

g) May have benefits of safety and health issues in mind; and

h) May be used in conjunction with preparations to provide relief from infection and/or inflammation.

It should also be understood that the term “comprise” where used herein is not to be considered to be used in a limiting sense. Accordingly, ‘comprise’ does not represent nor define an exclusive set of items, but includes the possibility of other components and items being added to the list.

This specification is also based on the understanding of the inventor regarding the prior art. The prior art description should not be regarded as being an authoritative disclosure of the true state of the prior art but rather as referring to considerations in and brought to the mind and attention of the inventor when developing this invention.

Aspects of the present invention have been described by way of example only and it should be appreciated that modifications and additions may be made thereto without departing from the scope thereof, as defined in the appended claims.

1. Protective aid, said protective aid including a body, said body having an internal and an external surface, said body adapted to include a defined cavity such that a substantial portion of the internal surface of the aid is distanced from an area being protected by the aid, said body also including an external rim contactable with a surface adjacent the area being protected, said protective aid also adapted to receive securing means on the body for securing the body relative to either or both the area being protected and a covering article.

2. Protective aid as claimed in claim 1 wherein the body of the protective aid is manufactured from breathable materials.

3. Protective aid as claimed in claim 2 wherein the body of the protective aid is manufactured from smooth, non-stick materials.

4. Protective aid as claimed in claim 3 wherein the body of the protective aid is manufactured from anti-allergenic, non-irritating materials.

5. Protective aid as claimed in claim 4 wherein the body of the protective aid is mouldable to a preferred shape.

6. Protective aid as claimed in claim 5 wherein the body of the protective aid is multi-layered.

7. Protective aid as claimed in claim 6 wherein the body includes a therapeutic layer.

8. Protective aid as claimed in claim 7 wherein the therapeutic layer is impregnated with, or adapted to receive a preparation directed to any one or more of facilitating healing, minimising infection, providing a cooling/anti-inflammatory effect.

9. Protective aid as claimed in claim 8 wherein the preparation is one from a list of sanitising, antiseptic, antimicrobial, anti-inflammatory preparations.

10. Securing means for use with a protective aid as claimed in claim 1, said securing means connectable to the protective aid and to either or both the area being protected and a covering article.

11. A method of manufacturing a protective aid, said protective aid including a body, said body having an internal and an external surface, said body adapted to include a defined cavity such that a substantial portion of the internal surface of the aid is distanced from an area being protected by the aid, said body also including an external rim contactable with a surface adjacent the area being protected, said protective aid also adapted to receive securing means on the body for securing the body relative to either or both the area being protected and a covering article, said method including the steps of:
a) Layering materials to produce a product of defined thickness and having preferred properties; and
b) Pressing or moulding said layered product to create a body having preferred dimensions; said body having a defined internal cavity, an outer perimeter/edge and an external and an internal surface; and relative to said external surface,
c) Including provision for receiving securing means.

12. A method of manufacturing a protective aid, said protective aid including a body, said body having an internal and an external surface, said body adapted to include a defined cavity such that a substantial portion of the internal surface of the aid is distanced from an area being protected by the aid, said body also including an external rim contactable with a surface adjacent the area being protected, said protective aid also adapted to receive securing means on the body for securing the body relative to either or both the area being protected and a covering article, said method including the steps of:
a) From a mouldable material having a defined thickness and having preferred properties;
b) Pressing or moulding said material to create a body having preferred dimensions; said body having a defined internal cavity, an outer perimeter/edge and an external and an internal surface; and relative to said external surface,
c) Including provision for receiving securing means.
